(19) United States
(12) Patent Application Publication
(10) Pub. No.: US 2005/0234380 A1
(43) Pub. Date: Oct. 20, 2005

(54) Proection pad for the trochantheric region and device comprising the pad

(75) Inventors: Robert Stephen Fulford, Bury (GB); George Edward Torrens, Nottingham (GB)

Correspondence Address: SUGHRUE MION, PLLC, 2100 PENNSYLVANIA AVENUE, N.W., SUITE 800, WASHINGTON, DC, 20037, US

(73) Assignee: REMPLOY LIMITED, Coventry (GB)

(21) Appl. No.: 10/520,880

(22) PCT Filed: Jul. 22, 2003
(86) PCT No.: PCT/GB03/03134

§ 371 Date: Jan. 11, 2005

(30) Foreign Application Priority Data
Jul. 24, 2002 (GB) 0217190.8

(51) Int. Cl. A61F 13/00; A61F 13/06

(52) U.S. Cl. 602/61

(57) ABSTRACT

When people fall they tend to fall on their side. This can lead to them breaking their femur particularly at the head around the trochantheric region. The head of the femur includes a portion called the greater trochanter which projects outwards and which is covered by very little flesh. A pad for use in protecting trochantheric region of the body comprises at least two separate layers, a substantially rigid layer, and a layer of dense, closed-cell resilient foam material. The substantially rigid layer includes a protecting boss which, in use, is located in the region of the greater trochanter to increase the spacing between the outside of the rigid layer and the greater trochanter. Preferably, the layer of closed-cell foam material extends over a greater area than the substantially rigid layer so that the periphery of the rigid layer lies entirely within the periphery of the foam layer.

### FIELD OF THE INVENTION

When people fall, particularly the elderly and frail or those less active whilst recovering from surgery, for example, they tend to fall on their side. This can lead to them breaking their femur particularly at the head around the trochantheric region. The head of the femur includes a portion called the greater trochanter which projects outwards and which is covered by very little flesh. When falling sideways onto this region, the femur can strike the ground and with very little flesh to cushion the impact, the femur is subjected to a considerable force which may be equivalent to as much as five times body weight acting on the greater trochanter. Since the inner end of the head of the femur is held substantially rigidly by the pelvis, the femur itself cannot move inwards to absorb the shock and this leads to the breakage of the femur in the trochantheric region.

### DISCUSSION OF THE PRIOR ART

Many proposals have been made to provide protection for this region for people who are particularly at risk and these usually involve placing some external padding or protective shell over the region of the greater trochanter. However, these often fail to absorb sufficient of the impact, or fail to distribute it over a sufficiently large area and so the resulting shear forces cause significant damage to the surrounding soft tissue. One example of a prior proposal is shown in WO 98/44817 which discloses an undergarment with pockets covering the trochantheric regions and protective pads arranged to be fitted into the pockets. The protective pads each comprise a substantially rigid outer shield with co-terminous inner layers of foam material. Another example of a prior proposal is shown in WO 95/19154 in which a domed shield is provided. The domed shield may have a substantially rigid plastics core surrounded by a foamed plastics material. A pair of domed shields are typically installed in an undergarment in positions to cover the trochantheric regions.

### SUMMARY OF THE INVENTION

According to a first aspect of this invention a pad for use in protecting the trochantheric region of the body comprises at least two separate layers, a substantially rigid layer and a layer of dense, closed cell resilient foam material; wherein the substantially rigid layer includes a projecting boss which, in use, is located in the region of the greater trochanter to increase the spacing between the outside of the rigid layer and the greater trochanter; and wherein the layer of closed-cell foam material extends over a greater area than the substantially rigid layer so that the periphery of the substantially rigid layer lies entirely within the periphery of the foam layer.

The projecting boss provides the first point of contact when a person wearing the pad falls sideways onto the ground. As a result of the boss providing the first point of contact the forces resulting from the fall can be dissipated, transferred and spread over the person's body in a controlled way to reduce the effect of the fall on the person. Any pressure or impact load on the boss of the substantially rigid layer is dissipated throughout the remainder of the rigid layer up to the periphery and is then cushioned by the foam layer. In this way the impact load is spread over a considerably greater area and so does not cause significant damage to the underlying soft tissue. This is especially so at the periphery of the substantially rigid layer since it is surrounded by the foam layer extending over a greater area.

Preferably, the side walls of the projecting boss are substantially frusto-conical so that the base of the boss extends over a greater area than the top. The inclined side walls contribute to spreading the impact load received by the top of the boss sideways and outwards and so, in use, away from the region of the greater trochanter. Preferably the side walls are flared outwards at the base to merge into the remainder of the substantially rigid layer and join the top of the boss at a rounded corner.

The top of the boss may be thinner than the remainder of the rigid layer with the sidewall of the boss having a thickness corresponding to that of the remainder of the substantially rigid layer at its base and tapering to that of the top of the boss at its top. By configuring the boss in this way it is possible to provide a so-called “crumple zone” on the outermost part of the boss with the deformation of the crumple zone absorbing a significant amount of the impact energy of any fall. This combination of features enables the high impact pressure applied to the top of the boss to be dissipated into a lower pressure spread over the remainder of the pad in a controlled way. The reduction in pressure reduces shear in the surrounding soft tissue and so reduces and substantially eliminates damage in this region.

Preferably a void is located beneath the boss either between the inside of the boss and the layer of foam, or beneath the layer of foam. This is especially important when the boss is configured to provide a crumple zone. The substantially rigid layer may be embedded in a closed cell foam material. In this case the closed cell foam material may be thinner on the outside of the top of the boss than on the remainder of the substantially rigid layer so that the boss is not apparent on merely looking at the external appearance of the pad. However, in this case, the presence of the boss can easily be located by feel which is useful for reasons which will be explained subsequently.

The substantially rigid layer may be formed of metal, particularly a low density metal such as aluminium or an aluminium alloy but preferably it is formed from a plastics material and is produced by moulding, preferably injection moulding. Polypropylene is a convenient and reasonably cheap material which is generally suitable but plain or filled nylon, polyurethane and other so-called “engineering plastics” may be used particularly where it is required to configure the shape and performance characteristics of the boss to provide a crumple zone. The foam layer may be formed from a closed cell, cross-linked low density polyethylene foam having a Shore hardness on the 00 scale of between 50 and 70 and preferably between 60 and 65.

The pad may include a third layer of less dense foam material remote from the substantially rigid layer. In this case it is preferred that the third layer has a greater extent than the layer of closed-cell foam and that the periphery of the layer of closed-cell foam lies wholly within the periphery of the third layer. The third layer further cushions and spreads any impact load. Equally since the periphery of the third layer extends outwards furthest and is the softest and thus most easily deformable, it merges into the shape and contour of the wearer's body so making the protective pad less obtrusive and providing wearing comfort.

Preferably, the third layer is formed of open cell foam material, such as PVC foam. Both foam layers may be formed from polyethylene or polyurethane but the closed cell foam may be formed from EVA. The or each foam layer preferably has a smooth outer appearance with edges that taper and merge into the wearer's body. Such tapering edges further enable pressure to be distributed in a controlled way to the body. The or each foam layer may be moulded, but it is simpler for the pieces to be die-cut from sheet material. The third layer may be formed from a conformable foam material. The rigid layer may be adhered to the closed cell foam layer and the closed cell foam layer adhered to the third layer. Of course when the substantially rigid layer is embedded within the closed cell foam layer adhesive is not usually required. Alternatively the rigid layer may be connected to the closed cell foam layer mechanically by stitching, stapling, riveting or use of a plastics connector.

The inside of the projecting boss may be partly filled with an additional layer of foam or a gel pad. This provides a greater degree of resilient padding over the greater trochanter region of the body where the femur is closest to the body surface and so affords greater protection.

Preferably, apertures are provided through at least the rigid and closed cell foam layers of a pad to provide for ventilation. Ventilation holes should also be provided through the third layer if the third layer includes closed cell material but, if the third layer is formed from open cell material it may not be necessary specifically to include ventilation holes. The ventilation holes may increase in size from the substantially rigid layer, through the closed cell foam layer to the third layer so that the layer closest to the wearer's skin includes the largest ventilation holes. By providing ventilation holes this improves the dissipation of heat and moisture from beneath the pad, in use, and so makes the wearing of the pad more comfortable for the user. A layer of wicking material such as a non-woven synthetic viscose known by the trade name of CAMBRELLE may be provided on the side of the pad adjacent the wearer to dissipate any perspiration.

Particularly when recovering from hip surgery it may be desirable either to heat the region beneath the pad to encourage greater blood flow and thereby encourage healing, or to cool the region beneath the pad, for example to reduce swelling. Accordingly, the pad may include a layer of material including carbon fibres or other resistive material on the side of the pad adjacent the patient, and a battery to provide a power supply to enable a small electrical current to be provided through the layer to heat it. To cool the region a refrigerated gel pad may be fitted inside the projecting boss. To facilitate this the projecting boss may be formed to be removable to enable the gel pad to be replaced without removal of the protection pad from the patient.

According to a second aspect of this invention a protection device for covering the trochantheric region of the body includes two protective pads in accordance with the first aspect of the invention connected to support means so that, in use, the pads cover the trochantheric regions of the user of the protection device.

The support means may include a strap which fits around the user's waist and, optionally, straps that fit around the user's thighs. In this case, the pads are attached to the strap at locations which, when the strap is worn around a user's waist, the pads, and particularly the raised bosses of the pads, lie over the region of the greater trochanter. It is preferred that the support means has the form of a garment which covers the trochantheric area and preferably has the form of an undergarment having a waistband and being arranged, in use, to extend down the thighs of the wearer. Preferably the waistband is elasticated. Preferably the garment comes in a number of standard sizes. Preferably the girth of the garment is adjustable so that the undergarment can be adjusted to fit the wearer precisely when the wearer is of a size which is intermediate that of the standard size undergarments. Typically the garment includes an adjustable front panel adjustable by about 100 mm.

The garment may be formed as a disposable garment and, for example, manufactured from a non-woven material. In this case it is preferred that the pads are permanently fixed to the garment, for example by stitching, mechanical fastening or adhesive. Alternatively, the garment may be intended to be a washable and reusable garment. In this case, the pads may be removable to facilitate washing and drying but typically the pads are themselves washable and capable of being tumble dried and, accordingly it is not necessary for the pads to be removable. It is preferred that the garment includes pockets which hold the pads. Preferably, garments of different size still include pockets of the same size to take commonly sized protective pads. Where the pads are held in pockets in the garment the pads may be held simply by the shape and size of the pocket or by additional fixing means such as, for example, stitching, snap fastenings or hook and loop type fastenings. When the garments do not include pockets the pads can of course be directly connected to the inside or outside of the garments by snap fastenings or hook and loop type fastenings, for example.

Many users of the protection device may also have difficulty in toileting. Accordingly, the garment may include a releasable gusset and/or be adapated to receive incontinence pads. The garment may also be designed to be washable and include a pad of absorbent material.

### BRIEF DESCRIPTION OF THE DRAWINGS

Particular examples in accordance with this invention will now be described with reference to the accompanying drawings; in which:

FIG. 1 is an exploded perspective view of components forming a first example of a pad;

FIG. 2 is a cross-section through the first example;

FIG. 3 is a longitudinal section through the first example;

FIG. 4 is a longitudinal section through a second example of pad;

FIG. 5 is an exploded perspective view of the components forming a third example of pad;

FIG. 6 is a plan view of a third example of pad;

FIG. 7 is a sectioned perspective view of a fourth example of pad;

FIG. 8 is a scrap longitudinal section through a modification of a pad;

FIG. 9 is a first example of a protection device including the pads;

FIG. 10 is a second example of protection device;

FIG. 11 is a third example of protection device; and,

FIG. 12 is a modification of the third example of protection device.

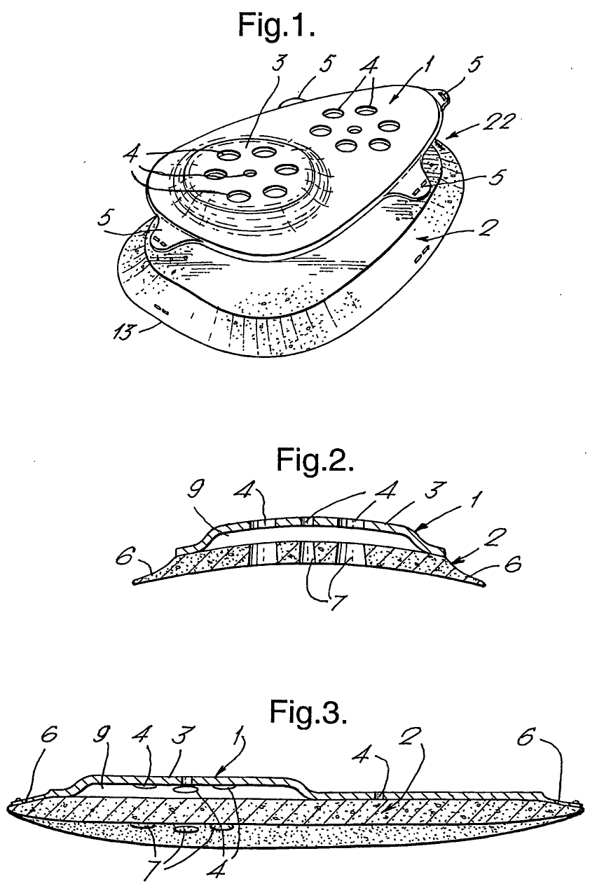

### DESCRIPTION OF PARTICULAR EXAMPLES

The first example of protective pad comprises a substantially rigid layer 1 and a layer of dense closed-cell resilient foam material 2. The substantially rigid layer 1 includes a projecting boss 3 and arrays of ventilation holes 4. The substantially rigid layer 1 also includes thin fixing lugs 5 projecting from its periphery. The dense closed-cell resilient foam layer 2 includes a chamfered outer peripheral region 6 and ventilation holes 7 which align with the ventilation holes 4 in the rigid layer 1. The ventilation holes 7 may be of larger diameter than the ventilation holes 4 or may be tapered as shown in FIGS. 2 and 3.

The substantially rigid layer 1 may be formed from polyurethane or polypropylene and the closed cell resilient foam layer 2 may be formed from polyurethane or, for example EVA foam. The two layers may be bonded together using an adhesive but in this example they are stitched together through the fixing lugs 5. The periphery of the substantially rigid layer 1 lies wholly within that of the closed cell foam layer 2. The raised boss 3 has a height of about 10 mm and a diameter of about 1001 mm. A void 9 is provided between the top of the raised boss 3 and the top of the layer of resilient closed cell foam 2.

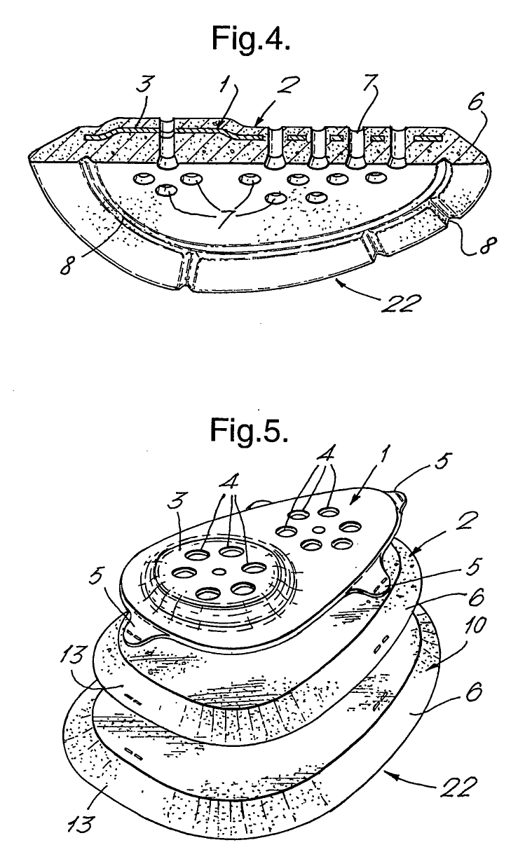

FIG. 4 shows a second example of protective pad in accordance with this invention in which the substantially rigid layer 1 is embedded within the layer 2 of closed cell resilient foam material. Again the substantially rigid layer 1 includes a raised boss 3 and, in this example, the outer profile of the pad mirrors that of the substantially rigid layer 1 and so also includes a raised boss. However, the thickness of the foam layer 2 over the top of the raised boss 1 may be less than that over the remainder of the substantially rigid layer so that the outer profile of the pad is substantially smooth. No fixing lugs 5 are required when the rigid layer 1 is embedded within the closed cell foam layer 2. A groove 8 is substantially aligned with the periphery of the rigid layer 1. This shaping of the foam layer 2 further helps spread any load on the patient resulting from the edge of the rigid layer 1.

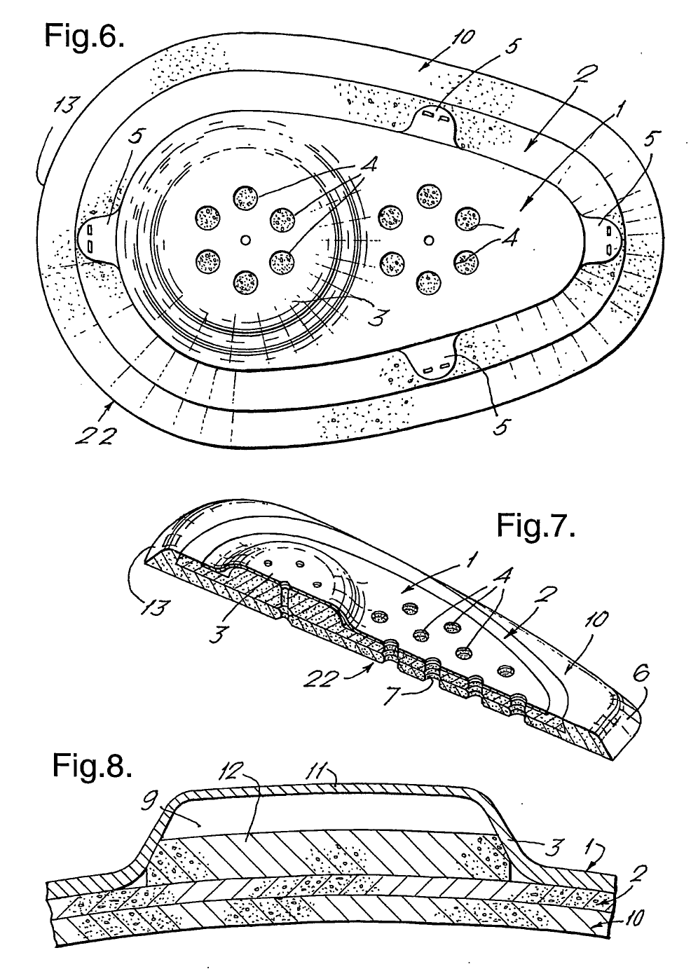

The third example of pad in accordance with this invention and, as shown in FIGS. 5 and 6 includes three separate layers. Firstly a substantially rigid layer 1, secondly a layer of closed cell resilient foam material 2 and thirdly a layer 10 of open cell resilient foam material 10. As in the first and second example the layer 2 is of greater extent than the layer 1 and layer 10 is also of greater extent than the layer 2 so that the periphery of layer 2 lies wholly within that of layer 10. This is shown most clearly in FIG. 6. In the third example the layers 2 and 10 are merely die cut from a sheet of suitable foam material with, effectively, steps formed between each layer. Again the layers are stitched together and each includes ventilation holes 7 (not shown in FIG. 5) which may be tapered, as shown in FIGS. 2 and 3 or be stepped with the largest holes being in layer 10. Again, in this example, a void is provided beneath the underside of the boss 3 and the upper side of the layer 2 similar to the void 9 shown in FIGS. 2 and 3.

FIG. 7 shows a fourth example of pad in accordance with this invention in which, again, three layers 1, 2 and 10 are provided similar to the third example but the layers are moulded to provide a profile with a chamfered edge 6 as shown in the first example. Again each layer 1, 2 and 10 is of greater extent than the preceding layer. In this example the layer 2 extends into and substantially fills the inside of the projecting boss 3.

FIG. 8 is a longitudinal section through a modification of the boss 3 showing how its wall thickness varies between a relatively thin wall thickness on a top portion 11 of the boss 3 and a relatively thick wall thickness on the remainder of the layer 1. FIG. 8 clearly shows how the wall thickness tapers from the base of the boss along its frustoconical sidewalls to its top. FIG. 8 also shows the provision of an additional layer 12 of resilient foam or gel material partly filling the inside of the boss 3 to provide additional resilience and therefore additional padding in this region.

By configuring the wall thickness of the boss 3 in the way shown in FIG. 8 it can be arranged that the outermost portion of the boss 3 provides a so-called “crumple zone” upon impact. This crumpling of the outermost portion of the boss 3 absorbs a significant amount of any impact force received by the boss 3. Since the frustoconical sidewalls of the boss 3 are tapered the boss 3 becomes more resistant to deformation as crumpling proceeds to provide a graduated transfer of any impact from the top 11 of the boss 3 to the remainder of the substantially rigid portion 1 and indeed to the remainder of the pad.

In use, the protective pads are oriented generally upright with the raised boss 3 at the top. The layers 1, 2 and 10 are curved in the transverse direction as shown most clearly in FIG. 2 so that they conform to the hip and thigh region of a user. The protective pads are located so that the boss 3 lies over the greater trochanter of the femur of the user. The top edge 13 of the pad is shaped to match the profile of human hips at the iliac crest of the pelvis. Thus the top edge of the pad sits over the muscle interface known as the gluteus medius, that is just below the iliac crest. The iliac crest has an approximate centre of rotation around the greater trochanter and thus, as can be seen in the drawings, the top edge 13 of the pad is approximately arcuate and centred on the centre of the boss 3. This correspondence between the pad and the iliac crest makes it easier to ensure that the protective pad is located at the correct position of the wearer and the location of the boss 3 can easily be located by feel to ensure that it is located in the correct position over the greater trochanter during fitting.

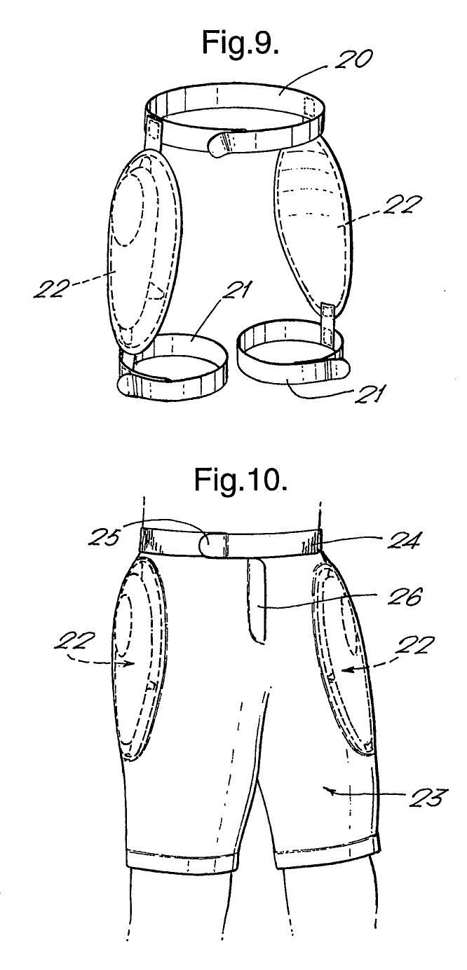

Whilst it is possible for the protective pads to be fixed to the wearer's body by the use of an appropriate medical adhesive applied to its inner surface or, via an intermediate double-sided medical adhesive tape, it is very much preferred that the pads are used with a specially designed support such as shown in FIGS. 9-12.

FIG. 9 shows a first example of protective device comprising a waist strap 20, a pair of leg straps 21, and a pair of pads 22, as previously described, fixed between the waist strap 20 and leg straps 21. The waist strap 20 is of adjustable length and, at least one of the pads 22 is movable along the waist strap 20 so that the support can be fixed to a wearer with the boss 3 of the pads 22 located over the greater trochanter of each leg. The leg straps 21 are also adjustable in length. The straps 20 and 21 may include buckles but preferably they include hook and loop type fastenings. The arrangement shown in FIG. 9 gives protection for the trochantheric region of the wearer whilst giving substantially unimpeded access to the wearer 20 by medical staff, for example. The device shown in FIG. 9 can be worn underneath, or on top of other garments.

This arrangement holds the pads 22 securely in the correct location whilst, at the same time, providing the least contact with the wearer's body.

A second example of support is shown in FIG. 10 and, this example is designed as a disposable, single-use, garment 23 made, preferably from a non-woven material having wicking properties. The entire garment may have some elasticity built in and it includes an elasticated waist band 24 with a front fastening 25. The pads 22 are sewn directly onto the garment 23 using, for example, the fixing lugs 5. The garment may include a fly-front 26 or a gusset which will be described subsequently.

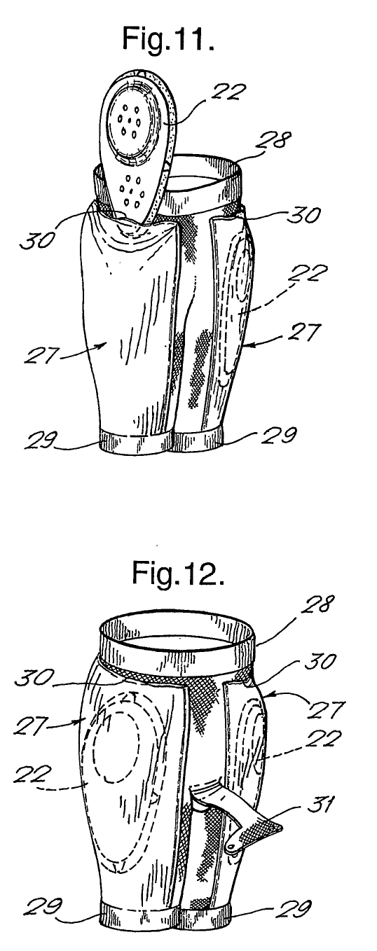

A third example is shown in FIG. 11 and comprises an undergarment 27 including an elasticated waistband 28 and elasticated cuffs 29 which fit around the thighs just above the knees. It also includes pockets 30 each sized to accommodate a pad 22. This garment is designed to be reusable and may be supplied as one of a number of garments, each of which is washable, and a single pair of pads 22 which can be transferred to the garment being worn at any particular instant. The undergarment 27 may include a fly front as shown in FIG. 10 or may include an openable gusset 31 as shown in FIG. 12. The undergarment 27 may be made from woven or non-woven material and the pockets may remain open at their upper end or may include a fastening, for example a hook and loop fastener, a zip fastener, a snap fastener or hook and eyelet fastener or be stitched closed to ensure that the pads 22 are retained in the pockets 30.

All of the garments described above typically come in a variety of sizes and each may include a central adjustable panel to enable it to be tailored to fit exactly around the waist of the user. Typically, the central adjustable panel includes a hook and loop type fastener and is adjustable up to a maximum of 100 mm.

1. A pad for use in protecting the trochantheric region of the body comprises at least two separate layers, a substantially rigid layer, and a layer of dense, closed-cell resilient foam material; wherein the substantially rigid layer includes a projecting boss which, in use, is located in the region of the greater trochanter to increase the spacing between the outside of the rigid layer and the greater trochanter; and wherein the layer of closed-cell foam material extends over a greater are than the substantially rigid layer so that the periphery of the substantially rigid layer lies entirely within the periphery of the foam layer.

2. A pad according to claim 1, in which the side walls of the projecting boss are substantially frusto-conical so that the base of the boss extends over a greater area than the top.

3. A pad according to claim 2, in which the side walls of the boss are flared outwards at the base to merge into the remainder of the substantially rigid layer and join the top of the boss at a rounded corner.

4. A pad according to claim 1, in which the top of the boss is thinner than the remainder of the rigid layer with the sidewall of the boss having a thickness corresponding to that of the remainder of the substantially rigid layer at its base and tapering to that of the top of the boss at its top.

5. A pad according to claim 1, in which the pad includes a third layer of less dense foam material remote from the substantially rigid layer.

6. A pad according to claim 5, in which the third layer has a greater extent than the layer of closed-cell foam and that the periphery of the layer of closed-cell foam lies wholly within the periphery of the third layer.

7. A pad according to claim 1, in which apertures are provided through at least the rigid and closed cell foam layers of a pad to provide for ventilation.

8. A protection device for covering the trochantheric region of the body includes two protective pads in accordance with claim 1, connected to a support so that, in use, the pads cover the trochantheric regions of the user of the protection device.

9. A protection device according to claim 8, in which the support has the form of a garment which covers the trochantheric area.

10. A protection device according to claim 9, in which the garment has the form of an undergarment having a waistband and being arranged, in use, to extend down the thighs of the wearer.

11. A protective device according to claim 9, in which the garment includes pockets which hold the pads.
